(19) United States
(12) Patent Application Publication
(10) Pub. No.: US 2021/0196637 A1
(43) Pub. Date: Jul. 1, 2021

(54) Compressible Cannabinoid Pharmaceutical Composition

(71) Applicant: Imbucanna, Inc., Coral Gables, FL (US)

(72) Inventor: Leo Mendez, Coral Gables, FL (US)

(21) Appl. No.: 17/127,186

(22) Filed: Dec. 18, 2020

Related U.S. Application Data
(63) Continuation of application No. PCT/US20/37464, filed on Jun. 12, 2020.
(60) Provisional application No. 62/861,122, filed on Jun. 13, 2019.

(51) Int. Cl. A61K 9/20; A61K 31/05

(52) U.S. Cl. CPC A61K 9/2054; A61K 9/2018; A61K 9/2031; A61K 31/352; A61K 9/2095; A61K 31/05; A61K 9/2027

(57) ABSTRACT

A compressible pharmaceutical composition comprising a cannabinoid and at least one excipient is disclosed. The composition may be an intermediate used in the manufacture of compressible dosage forms of cannabinoids such as tablets. The cannabinoid may be CBD or THC. The compressible excipient may be a material such as microcrystalline cellulose or lactose, or a matrix forming polymer such as a polyvinylpyrrolidone-vinyl acetate copolymer; a polyvinylcaprolactam, polyvinyl acetate, and polyethylene glycol 6000 copolymer; and an ethylene oxide and propylene oxide copolymer. Also disclosed are dry granulation processes for manufacturing the inventive composition, including slugging, roller compaction, hot-melt extrusion, and melt granulation.

### CROSS REFERENCE TO RELATED APPLICATIONS

This patent application claims priority to U.S. Patent applications 62/861,122 filed Jun. 13, 2019, and 62/876,754 filed Jul. 21, 2019, the contents of which are incorporated by reference.

### FIELD OF THE INVENTION

A compressible pharmaceutical composition comprising a cannabinoid and at least one excipient is disclosed. The composition may be used in the manufacture of compressible dosage forms of cannabinoids such as tablets.

### BACKGROUND

Many cannabinoids derived from natural cannabis plant material are of great interest currently for a variety of medical and recreational purposes. In particular, cannabidiol (CBD) has drawn great interest for a number of pharmacological effects, because CBD is not an intoxicant. CBD is currently be prescribed for indications including pain relief, as an anxiolytic, and appetite stimulant. Other cannabinoids are likewise being intensively investigated.

Cannabis is a genus of flowering plants that includes three different species, Cannabis sativa, Cannabis indica and Cannabis ruderalis. The term “Cannabis plant(s)” encompasses wild type Cannabis and also variants thereof, including Cannabis chemovars which naturally contain different amounts of the individual cannabinoids. For example, some Cannabis strains have been bred to produce minimal levels of THC, the principal psychoactive constituent responsible for the high associated with it and other strains have been selectively bred to produce high levels of THC and other psychoactive cannabinoids. Hemp is a strain of Cannabis sativa and produces almost exclusively CBD and little or no THC.

Cannabis plants produce a unique family of terpeno-phenolic compounds called cannabinoids, which produce the intoxication effect from consuming marijuana, which is orally active or can be smoked. There are 483 identifiable chemical constituents known to exist in the Cannabis plant, and at least 85 different cannabinoids have been isolated from the plant. The two cannabinoids usually produced in greatest abundance are cannabidiol (CBD) and Δ9-tetrahydrocannabinol (THC), but only THC is psychoactive.

The best studied cannabinoids include tetrahydrocannabinol (THC), cannabidiol (CBD) and cannabinol (CBN). Other cannabinoids include for example, cannabichromene (CBC), cannabigerol (CBG) cannabinidiol (CBND), Cannabicyclol (CBL), Cannabivarin (CBV), Tetrahydrocannabivarin (THCV), Cannabidivarin (CBDV), Cannabichromevarin (CBCV) Cannabigerovarin (CBGV), Cannabigerol Monomethyl Ether (CBGM).

The identification of cannabidiol (CBD) and delta-9-tetrahydrocannabinol (THC) as the active compounds of marijuana (Cannabis sativa) prompted extensive research in medicinal chemistry and the development of numerous cannabinoid analogs, a class of diverse terpenophenols derived from Cannabis sativa and synthetic chemical compounds, that interact with cannabinoid receptors on cells and repress neurotransmitter release in the brain (G Appendino et al., “Cannabinoids: occurrence and medicinal chemistry,” Curr Med Chem, 2011, 18, 1085-99, doi: 10.2174/092986711794940888; Massi Pet al., “Cannabidiol as potential anticancer drug,” Br J Clin Pharmacol, 2013, 75(2), 303-312, doi: 10.1111/j.1365-2125.2012.04298.x; Freeman TP et al., “Medicinal use of cannabis based products and cannabinoids,” BMJ. 2019 Apr. 4; 365:11141. doi: 10.1136/bmj.l1141).

Synthetic cannabinoids encompass a variety of distinct chemical classes: the cannabinoids structurally related to THC, the cannabinoids not related to THC, such as (cannabimimetics) including the aminoalkyl indoles, 1,5-diarylpyrazoles, quinolines, and arylsulfonamides, and eicosanoids related to the endocannabinoids. Any or all of these cannabinoids can be used in the present invention.

THC, more formally (−)-trans-Δ⁹-tetrahydrocannabinol, approved as a drug in many countries with the INN “Dronabinol,” is a naturally occurring compound and is the primary active ingredient in marijuana. Marijuana is dried hemp plant Cannabis sativa. The leaves and stems of the plant contain cannabinoid compounds (including dronabinol). Dronabinol has been approved by the Food and Drug Administration for the control of nausea and vomiting associated with chemotherapy and for appetite stimulation of patients suffering from wasting syndrome. Synthetic dronabinol is a recognized pharmaceutically active ingredient (API), but natural botanical sources of Cannabis rather than synthetic THC are also known in the art. Any or all of these cannabinoids can be used in the present invention.

Dronabinol is a light yellow resinous oil that is sticky at room temperature and hardens upon refrigeration. Dronabinol is insoluble in water and is formulated in sesame oil. It has a pKa of 10.6 and an octanol-water partition coefficient: 6,000: 1 at pH 7. After oral administration, dronabinol has an onset of action of approximately 0.5 to 1 hours and peak effect at 2 to 4 hours. Duration of action for psychoactive effects is 4 to 6 hours, but the appetite stimulant effect of dronabinol may continue for 24 hours or longer after administration.

CBD does not have the clinically undesirable (but recreationally desirable) psychotropic effects but is capable of inhibiting many effects of receptor ligands in the endocannabinoid system, which are responsible for the expression of THC's angiogenic and psychotogenic properties (Zuardi A W, “Cannabidiol: from an inactive cannabinoid to a drug with wide spectrum of action,” Rev Bras Psiquiatr, 2008, 30, 271-280, doi: 10.1590/S1516-44462008000300015). Despite its different pharmacological and behavioral effects, CBD shares many beneficial effects, including the capacity to act as an immunomodulator, with classic psychocannabinoids (Kozela E et al., “Cannabidiol has been shown to inhibit pathogenic T cells,” Br J Pharmacol, 2011, 163(7), 1507-1519, doi: 10.1111/j.1476-5381.2011.01379.x).

### Pharmacological Activity of Cannabinoids

Anti-Inflammatory Activities

Cannabis extracts and several cannabinoids have been shown to exert broad anti-inflammatory activities in experimental models of inflammatory central nervous system (CNS) degenerative diseases. While clinical use of many cannabinoids is limited by their psychotropic effects, phytocannabinoids like CBD, which are devoid of psychoactive activity, are potentially safe and therapeutically effective alternatives for the alleviation of neuroinflammation and neurodegeneration (Kozela E, Lev N, et al. “Cannabidiol inhibits pathogenic T cells, decreases spinal microglial activation and ameliorates multiple sclerosis-like disease in C57BL/6 mice,” Br J Pharmacol, 2011,163,1507-1519, doi: 10.1111/j.1476-5381.2011.01379.x). CBD exerts a wide range of anti-inflammatory properties and regulates cell cycle and function of various immune cells. These effects include suppression of humoral responses, such as release of cytokines, chemokines, growth factors, as well as suppression of immune cell proliferation, activation, maturation, migration, and antigen presentation (Raphel Mechoulam et al., “Cannabidiol—Recent Advances,” Chem Biodivers, 2007, vol. 4, 1678-1692, doi: 10.1002/cbdv.200790147)

Among the many types of neurodegenerative diseases in which inflammation is involved, multiple sclerosis (MS) is one of those clearly induced and driven by dysfunctional immune system activity. MS is a demyelinating disease which causes cytotoxic, degenerative processes, including inflammation, demyelination, oligodendrocyte cell death, and axonal degeneration. (Ribeiro R, Yu F, et al. This leads to neurological deficits and clinical symptoms of visual and sensory disturbances, motor weakness, tremor, ataxia, and progressive disability (Compston A, Coles A, “Multiple sclerosis,” Lancet, 2008, 372, 1502-1517, doi: 10.1016/S0140-6736(08)61620-7). There is currently no cure.(Yu F, et al., “Therapeutic potential of a novel cannabinoid agent C52 in the mouse model of experimental autoimmune encephalomyelitis,” Neuroscience, 2013, 254, 427-442, doi: 10.1016/j.neuroscience.2013.09.005). Several cannabinoids, including THC and CBD, exhibit anti-proliferative, anti-oxidative, and neuroprotective properties (Mechoulam R et al., “Cannabidiol—recent advances,” 2007, Chem Biodivers, 4, 1678-1692, doi: 10.1002/cbdv.200790147). Sativex® (GW Pharmaceuticals), the world's first pharmaceutical prescription medicine derived from the Cannabis plant, was launched in April 2005 for neuropathic pain relief in multiple sclerosis. It is a mixture of CBD and donabinol, and was most recently formulated as an oromucosal spray for the treatment of symptoms of spasticity associated with multiple sclerosis (G.W. Pharmaceuticals: Products and Pipeline, accessed at: https://www.gwpharm.com/products-pipeline/sativex, checked on Jul. 3, 2017).

Most current MS therapies are directed against various immune cells to achieve immunosuppressive effects, but immunosuppression alone is insufficient for therapeutic effect, especially in late, secondary, progressive MS, where neurodegenerative processes become resistant to immunomodulation (Bennett J L et al., “Update on inflammation neurodegeneration and immunoregulation in multiple sclerosis: therapeutic implications,” Clin Neuropharmacol, 2009, 32, 121-132, doi: 10.1097/WNF.0b013e3181880359.).

It appears that in this unique aspect, the endocannabinoid system could provide a rescue mechanism, particularly for patients suffering from late-stage MS. Research indicates that THC-like cannabinoids possess ameliorating, neuroprotective activity in this respect, and that cannabinoid-mediated neuroprotection, rather than immunosuppression, is relevant for the recovery process at the later, remissive stages of MS (Croxford J L et al., “Cannabinoid-mediated neuroprotection, not immunosuppression, may be more relevant to multiple sclerosis,” J Neuroimmunol, 2008, 193, 120-129, doi: 10.1016/j.jneuroim.2007.10.024; Maresz K et al., “Direct suppression of CNS autoimmune inflammation via the cannabinoid receptor CB1 on neurons and CB2 on autoreactive T cells”, Nat Med, 2007, 13, 492-497, doi: 10.1038/nm1561). CB1 receptors have been extensively studied and are implicated in a variety of potential CNS therapeutic effects. (S. Zou and U. Kumar, Cannabinoid Receptors and the Endocannabinoid System: Signaling and Function in the Central Nervous System, Int. J. Mol. Sci. 2018, 19, 833; doi:10.3390/ijms19030833).

Cancer

Cannabinoids may have two roles in the treatment of cancer. First, there is evidence of anticancer effects from several mechanisms. Second, cannabinoids may have a role in the palliative care for sequelae of cancer.

Several studies have demonstrated that cannabinoids exert an inhibitory action on the proliferation of various cancer cell lines and are able to slow down or arrest the growth of different models of tumor xenograft in experimental animals (Oesch S, Gertsch J., “Cannabinoid receptor ligands as potential anticancer agents—high hopes for new therapies?,” J Pharm Pharmacol. 2009, 61(7), 839-53, doi: 10.1211/jpp/61.07.0002; Alexander A et al., “Cannabinoids in the treatment of cancer,” Cancer Lett, 2009, 285(1), 6-12, doi: 10.1016/j.canlet.2009.04.005; Flygare Jet al., “The endocannabinoid system in cancer-potential therapeutic target?” Semin Cancer Biol, 2008, 18, 176-189 doi: 10.1016/j.semcancer.2007.12.008; Freimuth N et al., “Antitumorogenic effects of cannabinoids beyond apoptosis,” J Pharmacol Exp, 2010, Ther 332, 336-344, doi: 10.1124/jpet.109.157735; Guindon J et a., “The endocannabinoid system and cancer: therapeutic implication,” Br J Pharmacol, 2011, 163, 1447-1463, doi: doi: 10.1111/j.1476-5381.2011.01327.x). These data have attracted increasing interest for clinical exploitation of cannabinoid-based anti-cancer therapies.

Inhibition of cell proliferation by cannabinoids has also been studied. Modulation of cancer cell invasion has recently emerged as a topic of increasing interest (McAllister S D, Christian R T, et al., “Cannabidiol as a novel inhibitor of id-1 gene expression in aggressive breast cancer cells,” Mol Cancer, Ther, 2007, 6, 2921-7, doi: 10.1159/000370243; Blazquez C et al., “Cannabinoids inhibit glioma cell invasion by down-regulating matrix metalloproteinase-2 expression,” Cancer Res, 2008, 68, 1945-52, doi: 10.1158/0008-5472.CAN-07-5176; Ramer R et al., “Inhibition of cancer cell invasion by cannabinoids via increased expression of tissue inhibitor of matrix metal loproteinases-1,” J Natl Cancer Inst, 2008, 100, 59-69, doi: 10.1093/jnci/djm268).

Several cannabinoids have been shown to exert anti-proliferative and pro-apoptotic effects in various cancer types (lung, glioma, thyroid, lymphoma, skin, pancreas, uterus, breast, prostate, and colorectal carcinoma) (D. Wade et al., “A preliminary controlled study to determine whether whole-plant cannabis extracts can improve intractable neurogenic symptoms,” Clin. Reha, 2003, 17, 21-29, doi: 10.1191/0269215503cr581oa; T. J. Nurmikko et al., Pain , 2007,133, 210-220; Other antitumorogenic mechanisms are emerging, showing their ability to interfere with tumor neovascularization, cancer cell migration, adhesion, invasion and metastasization (McGivern J G, “Ziconotide: a review of its pharmacology and use in the treatment of pain,” Neuropsychiatr Dis. Treat, 2007, 3, 69-85, doi: 10.2147/nedt.2007.3.1.69).

The clinical use of THC and additional synthetic agonists is often limited by their unwanted psychoactive side effects, and for this reason, interest in non-psychoactive phytocannabinoids, such as CBD, has substantially increased in recent years. CBD does not have psychotropic activity and yet maintains very high potency. In 2006, CBD was used to selectively inhibit the growth of different breast tumor cell lines (MCF7, MDA-MB-231), while exhibiting lower potency in non-cancer cells (Ligresti A et al., “Antitumor activity of plant cannabinoids with emphasis on the effect of cannabidiol on human breast carcinoma,” J Pharmacol Exp Ther, 2006, 318, 1375-87, doi: 10.1124/jpet.106.105247).

CBD also possesses antitumor properties in gliomas, tumors of glial origin characterized by a high morphological and genetic heterogeneity and considered one of the most devastating neoplasms, showing high proliferative rate, aggressive invasiveness and insensitivity to radio- and chemo-therapy (Massi P et al., “Cannabidiol as potential anticancer drug,” Br J Clin Pharmacol , 2013, 75(2), 303-312, doi: 10.1111/j.1365-2125.2012.04298.x). Research findings have also suggested a novel mechanism underlying the anti-invasive action of CBD on human lung cancer cells, and imply its use as a therapeutic option for the treatment of invasive cancers, as well as leukemia (S. Goodin, Am. J. Health-Syst Pharm. 65 (2008) S 10eS 15;[£]F. Y. F. Lee, R. Borzilleri, C. R. Fairchild, et al., Cancer Chemother. Pharmacol. 63 (2008) 157el66; A. Conlin, M. Fornier, C. Hudis, et al., Eur. J. Cancer 44 (2008) 341 e352; D. R. P. Guay, Consult Pharm. 24 (2009) 210e226; N. Slatkin, J. Thomas, A. G. Lipman, et al., J. Support Oncol. 7 (2009) 39e46; F. M. Reichle, P. F. Conzen, Curr. Opin. Invest. Drugs 9 (2008) 90el00; C. S. Yuan, Ann. Pharmacother. 41 (2007) 984e993; M. D. Kraft, Am. J. Health Syst. Pharm. 64 (2007) S 13eS20; Novartis: Press release 30 Mar. 2009. Available at: http://www.novartis.com/ accessed Mar. 5, 2017; D. L. Higgins, R. Chang, D. V. Debabov, et al., Antimicrob. Agents Chemother. 49 (2005) 1 127el134; J. K. Judice, J. L. Pace, Bioorg. Med. Chem. Lett. 13 (2003) 4165e4168; S. Avaleeson, J. L. Kuti, D. P. Nicolau, Expert Opin. Invest. Drugs 16 (2007) 347e357).

In addition to their anti-proliferative and pro-apoptotic actions, it has been shown that cannabinoids can affect other important processes in tumorigenesis, in particular, angiogenesis, or the formation of new blood vessels from pre-existing ones—an essential step in tumor growth, invasion, and metastasis and a major therapeutic target for cancer therapy (Solinas M et al., “Cannabidiol inhibits angiogenesis by multiple mechanisms,” Brit J Pharmacol, 2012, 167, 1218-31, doi: 10.1111/j.1476-5381.2012.02050.x). Strategic approaches are needed that are aimed at the timely administration of natural, non-psychotropic cannabinoids (such as CBD) that are able to suppress pro-angiogenic factor production while binding with low affinity to cannabinoid receptors, thereby excluding psychotropic and immune or peripheral effects (Casanova M L et al., “Inhibition of skin tumor growth and angiogenesis in vivo by activation of cannabinoid receptors” J Clin Invest, 111, 43-50, doi: 10.1172/JC116116; Blazquez C, Gonzales Feria L et al., “Cannabinoids inhibit the vascular endothelial growth factor pathway in gliomas,” Cancer Res,2004, 64, 5617-5623, doi: 10.1158/0008-5472.CAN-03-3927; Gertsch J et al., “Phytocannabinoids beyond the Cannabis plant—do they exist?,” Br J Pharmacol, 2010, 160: 523-29, doi: 10.1111/j.1476-5381.2010.00745.x; Preet A et al., “Delta-9-tetrahydrocannabinol inhibits epithelial growth factor-induced lung cancer cell migration in vitro as well as its growth and metastasis in vivo,” Oncogene, 2007, 27, 339-346, doi: 10.1038/sj.onc.1210641; Russo E B, “Taming THC: potential Cannabis synergy and phytocannabinoid-terpenoid entourage effects,” Br J Pharmacol, 2011, 163, 1344-1364, doi: 10.1111/j.1476-5381.2011.01238.x).

Collectively, the non-psychoactive plant-derived cannabinoid CBD exhibits pro-apoptotic and anti-proliferative actions in different types of tumors and may also exert anti-migratory, anti-invasive, anti-metastatic, and perhaps anti-angiogenic properties. On this basis, evidence supports that CBD is a potent and selective inhibitor of cancer cell growth and spread (Massi P et al., “Cannabidiol as potential anticancer drug,” Br J Clin Pharmacol, 2013, 75(2), 303-312, doi: 10.1111/j.1365-2125.2012.04298.x). Considering its demonstrated clinical efficacy and safety in multiple sclerosis patients, the findings suggest that CBD is worthy of clinical consideration in an appropriate formulation for cancer therapy.

Cannabinoids are currently used in cancer patients to palliate wasting, emesis, and pain that often accompany cancer (Massi P et al., “Cannabidiol as potential anticancer drug,” Br J Clin Pharmacol, 2013, 75(2), 303-312, doi: 10.1111/j.1365-2125.2012.04298.x); J. R. Johnson et al., “Multicenter, double-blind, randomized, placebo-controlled, parallel-group study of the efficacy, safety, and tolerability of THC:CBD extract and THC extract in patients with intractable cancer-related pain,” J. Pain Symptom Manag., 2010, 39, 167-799 doi: 10.1016/j.jpainsymman.2009.06.008. A shortcoming for these and forthcoming indications clearly lies in the psychoactive adverse effects of cannabinoids, resulting in increased interest in the non-psychoactive cannabinoid CBD in recent years (Ramer R, Merkord J, et al., “Cannabidiol inhibits cancer cell invasion via upregulation of tissue inhibitor of matrix metal loproteinases-1”. Biochem Pharm, 2010, 79, 955-966, doi: 10.1093/jnci/djm268).

Mental Illness/Neuropathic Pain

Cannabinoids exert their pharmacological effects via cannabinoid receptors which are widely distributed in the central nervous system (Mackie K, “Cannabinoid receptors: where they are and what they do,” J Neuroendocrinol. 2008, 1, 10-4. doi: 10.1111/j.1365-2826.2008.01671.x). Several of the physiological effects

Spinal cord injury and neuropathic pain are diseases in which alterations in the endocannabinoid system have been demonstrated, paving the way for new therapeutic strategies in which normal endocannabinoid system functionality is restored (Pacher P et al., “The endocannabinoid system as an emerging target of pharmacotherapy,” Pharmacol Rev 2006, 58, 389-462, doi: 10.1124/pr.58.3.2] Sativex® was also approved for use in some countries as an adjunctive analgesic for severe pain in advanced cancer patients, reducing use of, and dependency on, opioid medications. It efficiently reduces pain in patients with advance cancer, and has been recommended by the FDA for direct entry into Phase III trials (Johnson J R et al., “Multicenter, double-blind, randomized, placebo-controlled, parallel-group study of the efficacy, safety, and tolerability of THC:CBD extract and THC extract in patients with intractable cancer-related pain,” J. Pain Symptom Manage, 2010, 39 (2), 167-79, doi: 10.1016/j.jpainsymman.2009.06.008).

Many studies present evidence that support the therapeutic potential of cannabidiol to mitigate the detrimental and psychotogenic effects of delta-9-tetrahydrocannabinol, mitigating its effects of acute induction of psychotic and anxiety systems (Bhattacharyya S et al., “Opposite effects of THC and CBD on human brain function and psychopathology,” Neuropsychopharmacol, 2009, 35, 764-774, doi: 10.1038/npp.2009.184; Hagerty S L et al., “The Cannabis conundrum: Thinking outside the THC box,” J Clin Pharmacol, 2015, 55(8), 839-41, doi: 10.1002/jcph.511).

Where THC has anxiogenic effects, CBD reduces subjective anxiety, achieving clinically significant reductions in anxiety, cognitive impairment, and discomfort. (Fusar-Poli P et al., “Distinct effects of delta-9-tetrahydrocannabinol and cannabidiol on neural activation during emotional processing” Arch Gen Psychiat, 2009, 66, 95-105, doi: 10.1001/archgenpsychiatry.2008.519; Bergamaschi M M et al., “Cannabidiol reduces the anxiety induced by simulated public speaking in treatment-naive social phobia patients” Neuropsychopharmacol, 2011, 36, 1219-1226, doi: 10.1038/npp.2011.6). Evidently, the effects of Cannabis are complex and arise from myriad cannabinoids; they are distinct from the artificially uniform effect of THC alone. The therapeutic effects of these cannabinoids can be harnessed most effectively through formulations which account for the complexity of cannabinoids and their interactions, and the mechanisms that underlie different effects of use in humans (i.e., cognitive effects versus anxiolytic or anxiogenic effects) (Hagerty S L et al., “The Cannabis conundrum: Thinking outside the THC box” J Clin Pharmacol, 2015, 55(8), 839-41, doi: 10.1002/jcph.511).

Epilepsy

Epilepsy is a prevalent and devastating disorder of the CNS, which may be defined as a brain condition causing spontaneous recurrent seizures. These seizures are sometimes both progressively severe and accompanied by cognitive and behavioral comorbidities (Goldberg E M et al., “Mechanisms of epileptogenesis: a convergence on neural circuit dysfunction,” Nat. Rev. Neurosci. 2013, 14, 337-49, doi: 10.1038/nrn3482).

Epileptogenesis (latency period) refers to a scantily understood cascade of events that generally transmute a non-epileptic brain into one that triggers spontaneous seizures; these events occur in a specific time window included between a brain-damaging insult such as stroke, infection, or genetic predisposition, and the onset of unprovoked and unpredictable seizures Ibid.) During this period, a specific treatment may stop or modify the epileptogenic process and thereby positively influence the quality of life of an epileptogenic subject (White H S, Loscher W. Searching for the ideal epileptogenic agent in experimental models: single treatment versus combinatorial treatment strategies. (2014) Neurotherapeutics 1 1: 373-384; Citraro R et al., “Antidepressants but not antipsychotics have antiepileptogenic effects with limited effects on comorbid depressive-like behavior in the wag/rij rat model of absence epilepsy,” Br J Pharmacol, 2015, 172, 3177-3188, doi: 10.1007/s13311-013-0250-1).

Currently, among the major unmet needs in the treatment of epilepsy there is the identification of disease-modifying drugs that can completely prevent epilepsy or slow its progression (Leo A, Russo E et al., “Cannabidiol and epilepsy: rationale and therapeutic potential,” Pharma Res, 2016, 107, 85-92, doi: 10.1016/j.phrs.2016.03.005). Unfortunately, many new antiepileptic drugs (AEDs) as well as older AEDs present solely symptomatic features, and do not possess antiepileptogenic or disease modifying features, and show several negative side effects influencing quality of life as much as seizures (Kwan P et al., “Refractory epilepsy: mechanisms and solutions,” Expert Rev Neurother, 2006, 6, 397-406, doi: 10.1586/14737175.6.3.397)

(Perucca P et al., “Adverse effects of antiepileptic drugs,” Lancet Neurol, 2012, 11, 792-802, doi: 10.1016/S1474-4422(12)70153-9).

Emphasis has been placed on phytocannabinoids, which have demonstrated clinically significant antiseizure effects in clinical trials (Reddy D S et al., “The pharmacological basis of Cannabis therapy for epilepsy,” J Pharmacol Exp Ther, 2016, 357(1), 45-55, doi: 10.1124/jpet.115.230151). Anecdotal reports indicate mixed findings for seizure prevalence subsequent to administration of THC, however, where a greater prevalence of grand mal seizures may be observed subsequent to consumption in previously seizure-free patients (Ramsey H H et al., “Anti-epileptic action of marijuana-active substances,” Fed Proc 8, 1949, 12(4), 747-748, doi: 10.1007/s13311-015-0375-5; Consroe P F et al., “Anticonvulsant nature of marihuana smoking,” JAMA, 1975, 234, 306-307, doi: 10.1001/jama.1975.03260160054015).

Studies have also suggested that desired therapeutic effects can be achieved via administration of pure CBD in twice-daily dosages in pediatric patients, resulting in seizure reduction ranging from total seizure freedom to reductions of 25-80%, and the absence of deleterious side effects (Saade D et al., “Pure cannabidiol in the treatment of malignant migrating partial seizures in infancy: A case report,” Pediatr. Neurol, 2015, 52, 544-47, doi: 10.1016/j.pediatrneurol.2015.02.008; Porter B E et al., “Report of a parent survey of cannabidiol-enriched Cannabis use in pediatric-resistant epilepsy,” Epilepsy Behav, 2013, 29, 574-77, doi: 10.1016/j.yebeh.2013.08.037). Thus, CBD or THC formulations may offer significant advantages over and above existing antiepileptic drugs (AEDs), accompanied by a superior pharmacokinetic, pharmacodynamic, and side effects profile. CBD or formulations may have the additive value of simultaneously managing psychiatric comorbidities associated with epilepsy, which are often more harmful to patients than seizures themselves (Dos Santos R G et al., “Phytocannabinoids and epilepsy” J Clin Pharm Ther, 2015, 40, 135-43, doi: 10.1111/jcpt.12235; Scuederi C et al., “Cannabidiol in medicine: a review of its therapeutic potential in ens disorders,” Phytother. Res, 2009, 23, 597-602, doi: 10.1002/ptr.2625). CBD or THC formulations may also be of additional value for drug-resistant epilepsies which are non-responsive to conventional AEDs (Leo A et al., “Cannabidiol and epilepsy: rationale and therapeutic potential,” Pharma Res, 2016, 107, 85-92, doi: 10.1016/j.phrs.2016.03.005,).

Mood Disorders

High CBD intake relative to THC has been associated with lower scores on the positive dimensions of the CAPE (Community Assessment of Psychic Experiences) Scale, inhibition of psychotic symptoms, and reduced deficiencies in episodic memory (Englund A et al., “Cannabidiol inhibits THC-elicited paranoid symptoms and hippocampal-dependent memory impairment,” J Pharmacol, 2013, 27(1), 19-27, doi: 10.1177/0269881112460109). CBD or THC formulations may have the advantage of an intentional dosage of synthetic or natural preparations with ideal pharmacology, to the exclusion of other cannabinoids and their respective pharmacokinetic and pharmacodynamics influences. The “protective effects” afforded by extended or sustained release formulations can be harnessed by virtue of the present invention, along with the desired therapeutic effects of cannabidiols like CBD, either alone or in concert with therapeutically effective amounts of other cannabinoids. (Englund A et al., “Cannabidiol inhibits THC-elicited paranoid symptoms and hippocampal-dependent memory impairment,” J Pharmacol, 2013, 27(1), 19-27, doi: 10.1177/0269881112460109).

Research has also indicated that the endocannabinoid system is intricately involved in the pathophysiology of depression, with CB1 receptors widely distributed in brain areas related to affective disorders, where expression is otherwise regulated by anti-depressants (Devane W A et al., “Determination and characterization of a cannabinoid receptor in rat brain,” Mol Pharmacol, 1988, 34, 605-613; Hill M N et al., “Is there a role for the endocannabinoid system in the etiology and treatment of melancholic depression?,” Behav Pharmacol, 2005, 16, 333-352; Hill M N et al., “Regional alterations in the endocannabinoid system in an animal model of depression: effects of concurrent antidepressant treatment,” J Neurochem, 2008, 106, 2322-36, doi: 10.1111/j.1471-4159.2008.05567).

Administration of CBD achieves characteristic effects of induced anti-psychotic and anxiolytic activity in subjects, and also attenuates the development of stress-induced behavior al consequences, raising the possibility that CBD could be useful for treating psychiatric disorders thought to involve impairment of stress-coping mechanisms, such as depression (Guimaraes F S et al., “Antianxiety effect of cannabidiol in the elevated plus-maze. Psychopharmacology,” 1990, 100, 558-559; Resstel L B et al., “Effects of cannabidiol and diazepam on behavior al and cardiovascular responses induced by contextual conditioned fear in rats,” Behav Brain Res, 2006, 172(2), 294-98, doi: 10.1016/j.bbr.2006.05.016; Resstel LB et al., “5-HT1A receptors are involved in the cannabidiol-induced attenuation of behavior al and cardiovascular-induced attenuation of behavior al and cardiovascular responses to acute restraint stress in rats,” Br J Pharmacol, 2009, 156(1), 181-88, doi: 10.1111/j.1476-5381.2008.00046.x; Zuardi A W et al., “Action of cannabidiol on the anxiety and other effects produced by delta 9-THC in normal subjects,” Psychopharmacology, 1982, 76, 245-50.

Studies indicate that CBD has a favorable profile in a model predictive of antidepressant-like activity in comparison to prototype antidepressants, but that such effects are only attainable at precise dosages, with smaller or higher doses producing no effect (Zanelati T V et al., “Antidepressant-like effects of cannabidiol in mice: possible involvement of 5-HT1A receptors,” Br J Pharmacol, 2010, 159, 122-28, doi: 10.1111/j.1476-5381.2009.00521.x). The present invention can be the basis for formulations capable of providing dosages in accordance with the most clinically appropriate pharmacokinetic and pharmacodynamic profile in order to achieve desired therapeutic effects in a patient presenting with a specific pathophysiology, such as major depressive disorder.

Sleep

Several studies report that THC affects sleep patterns (Pivik, R. T et al., “D-9-tetrahydrocannabinol and synhexl: effects on human sleep patterns.,” Clin. Pharmacol, 1972, Ther. 13 (3), 426-435, doi: 10.1002/cpt1972133426; Feinberg et al., “Effects of high dosage D-9-tetrahydrocannabinol on sleep patterns in man.,” Clin. Pharmacol, 1975, Ther. 17 (4), 458-466, doi: 10.1002/cpt1975174458; Feinberg et al., “Effects of marijuana extract and tetrahydrocannabinol on electroencephalographic sleep patterns.” Clin. Pharmacol, 1976, Ther. 19 (6), 782-794, doi: 10.1002/cpt1976196782). The chemistry of CBD has been examined, and its central nervous system (CNS) pharmacological properties, including its anticonvulsant, anxiolytic, and sedative effects, have been documented (Chesher et al., “Interaction of D\hat{ }-tetrahydrocannabinol and cannabidiol with phenobarbitone in protecting mice from electrically induced convulsions.,” J. Pharm. Pharmacol, 1975, 27 (8), 608-609, doi: 10.1111/j.2042-7158.1975.tb09515.x; Pickens et al., “Sedative activity of Cannabis in relation to its D\hat{ }-trans-tetrahydrocannabinol and cannabidiol content.,” Br. J. Pharmacol. 1981, 72 (4), 649-656; Russo. E et al., “ A tale of two cannabinoids: the therapeutic rationale for combining tetrahydrocannabinol and cannabidiol.,” Med. Hypotheses, 2006, 66 (2), 234-246, doi: 10.1016/j. mehy.2005.08.026).

In studies, it has been shown that CBD improves sleep in individuals suffering from insomnia (Carlini E A et al., “Hypnotic and antiepileptic effects of cannabidiol,” J. Clin. Pharmacol. 1981, (suppl 8-9), 417S-427S, doi: 10.1002/j.1552-4604.1981.tb02622.x). It has been successfully employed to block anxiety-induced REM sleep alteration via its anxiolytic effects (Hsiao Y et al., “Effect of cannabidiol on sleep disruption induced by the repeated combination tests consisting of open field and elevated plus-maze in rats,” Neuropharmacol, 2012, 62, 373-84, doi: 10.1016/j.neuropharm.2011.08.013). Other studies have exhibited clinically significant improvements in sleep in subjects suffering from post-traumatic stress-related insomnia, even when subjects received no pharmaceutical medications to treat sleep disorders aside from cannabidiol oil (Shannon S et al., “Effectiveness of cannabidiol oil for pediatric anxiety and insomnia as part of posttraumatic stress disorder: a case report,” Perm J, 2016, 20(4), 16-005, doi: 10.7812/TPP/16-005). In other studies, the systemic acute administration of CBD appears to increase total sleep time in subjects, in addition to increasing sleep latency in the light period of the day of administration (Chagas M et al., “Effects of acute systemic administration of cannabidiol on sleep-wake cycle in rats” J. Psychopharmacol, 2013, 27(3), 312-16, doi: 10.1177/0269881112474524).

### Addiction and the Endocannabinoid System

Drug addiction is a chronic, relapsing disease characterized by the compulsion to seek and take a drug, loss of control in limiting intake and emergence of negative emotional states when access to the drug is prevented (koob et al., “Drug abuse: hedonic homeostatic dysregulation,” Science, 1997, 278, 52-58, doi: 10.1126/science.278.5335.52) It is a chronic disorder involving persistent changes in the central nervous system.

Prototypical examples of those changes include tolerance, dependence, and/or sensitization after repeated drug exposure with corresponding neurochemical changes in the brain (Chao and Nestler, Molecular neurobiology of drug addiction (2004) Annu Rev Med. 55: 1 13-132; Nestler, Molecular mechanisms of drug addiction (2004) Neuropharm. 47:24-32; Ron and Jurd, The ‘ups and downs’ of signaling cascades in addiction (2005) Sci STKE 309:rel4). It is these neuropharmacological and neuroadaptive mechanisms that mediate the transition from occasional, controlled drug use to the loss of behavioral control over drug-seeking and drug-taking that defines addiction.

These emotional, cognitive and behavioral effects are commonly linked to a neurobiological subtract. The endocannabinoid system is strongly implicated in these neuroadaptations, which are induced through repeated exposure to drugs of abuse (Fattore et al., “Endocannabinoid system and opioid addiction: behavioral aspects,” Pharmacol Biochem Behav,2005, 81, 343-359, doi: 10.1016/j.pbb.2005.01.031). Such findings include the main legal and illegal drugs used in developed countries: nicotine, alcohol, cannabis, cocaine and opioids (Arnold, “The role of endocannabinoid transmission in cocaine addiction,” Pharmacol Biochem Behav, 2005, 81, 396-406, doi: 10.1016/j.pbb.2005.02.015; Colombo et al, “Endocannabinoid system and alcohol addiction: pharmacological studies,” Pharmacol Biochem Behav, 2005, 81, 369-380, doi: 10.1016/j.pbb.2005.01.022; Lopez-Moreno et al., “Functional interactions between endogenous cannabinoid and opioid systems: focus on alcohol, genetics and drug-addicted behaviors,” Curr Drug Targets, 2010, 11, 406-428, doi: 10.2174/138945010790980312; Maldonado et al., “Endogenous cannabinoid and opioid systems and their role in nicotine addiction,” Curr Drug Targets, 2010, 11, 440-449, doi: 10.2174/138945010790980358; Maldonado et al., “Involvement of the endocannabinoid system in drug addiction,” Trends Neurosci, 2006, 29, 225-232, doi: 10.1016/j.tins.2006.01.008; Piomelli, “The endogenous cannabinoid system and the treatment of marijuana dependence,” Neuropharmacology, 2004, 47(Suppl 1), 359-367, doi: 10.1016/j.neuropharm.2004.07.018).

The complexity of the endocannabinoid system is reflected by its implication in many different cognitive and physiological processes. It participates in the regulation and modulation of learning and memory, food intake, nociception, motor coordination, reward processes, emotional control, and various cardiovascular and immunological processes (Ameri, “The effects of cannabinoids on the brain,” Prog Neurobiol,1999, 58, 315-348, doi: 10.1016/S0301-0082(98)00087-2). The participation of the endocannabinoid system in most of these functional psycho-psychological processes is explained by its strong connection to the dopaminergic system, mainly through the basal ganglia and corticolimbic brain structures (Freund et al., “Differences in norepinephrine clearance cerebellar slices from low-alcohol-sensitive and high-alcohol sensitive rats”, Alcohol, 2003, 30, 9-18, doi: 10.1016/S0741-8329(03)00098-3).

The main excitatory and inhibitory systems of the mammalian central nervous system are under the influence of the endocannabinoid system. In the addicted individual, the imbalance in glutamatergic neurotransmission is common. It is also known that a dysregulation of excitatory signaling could lead to the relapse of drug use and cravings, supporting the notion of addictive behavior as a chronic disorder (Dackis et al., “Glutamatergic agents for cocaine dependence,” Ann N Y Acad Sci, 2003, 1003, 328-345, doi: 10.1196/annals.1300.021). Therefore, it is easy to understand the importance of the endocannabinoid system in the phenomenon of addiction, especially when its neuromodulation is compromised, for example, by an altered performance of receptors and cellular signaling of cannabinoid CB I receptors (Lopez-Moreno et al., “The pharmacology of the endocannabinoid system: functional and structural interactions with other neurotransmitter systems and their repercussions in behavioral addiction,” Addiction Biol, 2008, 13, 160-187, doi: 10.1111/j.1369-1600.2008.00105.x).

The endocannabinoid system is the major player and a neurobiological mechanism underlying drug reward (Onaivi, “An endocannabinoid hypothesis of drug reward and addiction”, Acad sci, 2008, 1139: 412-21, doi: 10.1196/annals.1432.056). The endocannabinoid system is a modulator of dopaminergic activity in the basal ganglia, elucidating its participation in the primary rewarding effects of alcohol, opioids, nicotine, cocaine, amphetamine, cannabinoids, and benzodiazepines through the release of endocannabinoids that act as retrograde messengers to inhibit classical transmitters, including dopamine, serotonin, GABA, glutamate, acetylcholine, and norepinephrine (Onaivi, “An endocannabinoid hypothesis of drug reward and addiction”, Acad sci, 2008, 1139: 412-21, doi:10.1196/annals.1432.056). The endocannabinoid system is further involved in the common mechanisms underlying relapse to drug-seeking behavior by mediating the motivational effects of drug-related environmental stimuli and drug re-exposure (Maldonado et al., “Involvement of the endocannabinoid system in drug addiction” Trends Neurosci, 2006, 29, 225-232, doi: 10.1016/j.tins.2006.01.008) The endocannabinoid system triggers or prevents reinstatement of drug-seeking behavior (Fattore et al., “An endocannabinoid mechanism in relapse to drug seeking: a review of animal studies and clinical perspectives”, Brain Res Rev, 2007, 53, 1-16, doi: 10.1016/j.brainresrev.2006.05.003).

The perturbation of the endocannabinoid system by drugs of abuse can be ameliorated byrestoring the perturbed system using cannabinoid receptor ligands. Cannabinoid receptor antagonists are useful in the reduction of drug use, in smoking cessation, and reduction in alcohol consumption, and rimonabant has been demonstrated to have antagonistic activity against disruption of cognition or reward-enhancing properties of morphine, amphetamine, cocaine, (Poncelet, Blockade of CB1 receptors by 141716 selectively antagonizes drug-induced reinstatement of exploratory behavior in gerbils (1999) Psychopharmacology 144: 144-50) ethanol, and diazepam. The blockade of the behavioral aversions by cannabinoid anatagonists after chronic administration of alcohol, cocaine, and diazepam is in agreement with data obtained during cannabinoid-induced alterations in brain dispositions of drugs of abuse that correlated with behavioral alterations in mice (Reid and Bornheim, “Cannabinoid-induced alterations in brain disposition of drugs of abuse,” Biochem. Pharmacol, 2001, 61, 1357-1367, doi: 10.1016/s0006-2952(01)00616-5). [0056] As the mesolimbic dopaminergic system is implicated in the reinforcing properties of most drugs of abuse, the endocannabinoid system is a therapeutic target for individuals addicted to drugs. Mice treated with CB1 antagonists (i.e. SRI 41716) showed a significant reduction in self-administered alcohol consumption (Colombo et al., “Suppressing effect of the cannabinoid CB1 receptor antagonist, SR 141716, on alcohol's motivational properties in alcohol-preferring rats,” Eur J Pharmacol, 2004, 498, 119-123, doi: 10.1016/j.ejphar.2004.07.069), cocaine-related locomotor activity (Gerdeman et al., “Context-specific reversal of cocaine sensitization by the CB1 cannabinoid receptor antagonist rimonabant,” Neuropsychopharmacology, 2008, 33, 2747-2759, doi: 10.1038/sj.npp.1301648), and a reduction in the reward effects of nicotine (Cohen et al., “SRI 41716, a central cannabinoid (CB(1)) receptor antagonist, blocks the motivational and dopamine-releasing effects of nicotine in rats”, Behav Pharmacol, 2002, 13, 451-63, doi:).

The inhibition of FAAH (e.g. by URB597) causes a reduction of nicotine-induced dopamine activity in the nucleus accumbens, leading to a reduction in nicotine-induced reinstatement of nicotine seeking (Forget et al., “Inhibition of fatty acid amide hydrolase reduces reinstatement of nicotine seeking but not break point for nicotine self-administration—comparison with CB1 receptor blockade,” Psychopharmacology (Berl), 2009, 205, 613-624, doi: 10.1007/s00213-009-1569-5).

Thus, the endocannabinoid physiological control system is a directly important natural regulatory mechanism for reward in the brain, and also contributes to reduction in aversive consequences of abused substances, such that manipulating the endocannabinoid system can be exploited in order to treat alcohol and drug dependency, and to reduce the behavioral consequences associated with withdrawal (Onaivi, “An endocannabinoid hypothesis of drug reward and addiction,” Acad scie, 2008, 1139, 412-21, doi: 10.1196/annals.1432.056).

Opioid Addiction

Abuse of heroin and prescription opioids have long constituted a significant burden to society both through the direct and indirect consequences of illicit opioid use. Since the mid-1990's heroin use has experienced a resurgence, particularly among younger populations. In 2004, an estimated 3.7 million people in the United States had reported using heroin at some point in their lifetime according to data collected by the National Institute on Drug Abuse. The 2008 National Survey on Drug Use and Health determined that the number of heroin users over the age of 12 in the United States had increased dramatically from 153,000 in 2007 to 213,000 in 2008.

The high abuse liability of heroin was demonstrated in a 2004 study of drug use, which found that 67% of those that used heroin also met the criteria for abuse or dependence, a statistic markedly higher than that for other drugs of abuse such as cocaine, marijuana, or sedatives. Heroin use, while extremely problematic, is restricted to a small percentage of the population. However, abuse of prescription opioids has become more prevalent with rates of use rapidly increasing, and now represents a serious public health issue. The misuse or abuse of prescription drugs occurs when a person takes a prescription drug that was not prescribed or taken in one dose or for reasons other than those prescribed. Abuse of prescription drugs can produce serious health effects, including addiction. The classes of prescription drugs that are commonly abused include include oral narcotics such as hydrocodone (Vicodin), oxycodone (OxyContin), propoxyphene (Darvon), hydromorphone (Dilaudid), meperidine (Demerol) and diphenoxylate (Lomotil), and their non-medical use has increased dramatically in recent years. For example, in 1990, the number of individuals initiating abuse of prescription opioids was 573,000. By the year 2000, the number had risen to over 2.5 million according to the National Institutes of Health. In 2009, for the first time, the number of individuals initiating prescription opioid use nearly equaled that of marijuana; a previously unprecedented and alarming finding. Concurrently, emergency department visits due to complications from non-medical use of hydrocodone and oxycodone rose by 170% and 450% respectively from 1994 to 2002. Furthermore, opioid-related deaths rose by more than 300% between 1999 and 2006 (OAS, 2009).

Similarly, withdrawal from opiates, such as heroin or oral narcotics, is characterized by a host of aversive physical and emotional symptoms. High rates of relapse and limited treatment success rates for opiate addiction have prompted a search for new approaches. Research over the past decade has shed light on the influence of endocannabinoids on the opioid system. Evidence from both animal and clinical studies show an interaction between these two systems, and targeting the EC system as provided by the instant invention provides a novel intervention strategy for managing opiate dependence and withdrawal.

Opioids, such as heroin and morphine, exert their physiological and behavioral effects through specific interactions with opioid receptors (Kieffer, “Opioids: first lessons from knockout mice,” Trends Pharmacol Sci, 1999, 20, 19-26, doi: 10.1016/S0165-6147(98)01279-6; Matthes et al., “Loss of morphine-induced analgesia, reward effect and withdrawal symptoms in mice lacking the mu-opioid-receptor gene,” Nature, 1996, 383, 819-823, doi: 10.1038/383819a0) CB1 and μ-receptors are similarly expressed in many brain areas involved in reward processes (Herkenham et al., “Characterization and localization of cannabinoid receptors in rat brain: a quantitative in vitro autoradiographic study,” J Neurosci, 1991, 11, 563-583, doi: 10.1523/JNEUROSCI.11-02-00563; Matsuda et al., “Localization of cannabinoid receptor mRNA in rat brain,” J Comp Neurol, 1993, 327(4), 535-550, doi: 10.1002/cne.903270406) These receptors share common signaling cascades (Howlett, “The cannabinoid receptors. Prostaglandins Other Lipid Mediat,” Elsevier, 2002, 68-69, 619-631, doi: 10.1016/S0090-6980(02)00060-6) There is a functional interaction between the endogenous cannabinoid and opioid systems (Manzanares et al., “Pharmacological and biochemical interactions between opioids and cannabinoids,” Trends Pharmacol Sci, 1999, 20(7), 287-294, doi: 10.1016/S0165-6147(99)01339-5).

Studies have demonstrated that under certain circumstances, cannabis use can be associated with positive treatment prognosis among opioid-dependent cohorts. For example, Epstein and Preston found that cannabis abuse and dependence were predictive of decreased heroin and cocaine use during treatment (Epsteinet al., “Does cannabis use predict poor outcome for heroin- dependent patients on maintenance treatment? Past findings and more evidence against,” Addiction, 2003, 98, 269-279, doi: 10.1046/j.1360-0443.2003.00310.x).

There is a growing body of literature suggesting that increased regulated access to medical and recreational cannabis can result in a reduction in the use of and subsequent harms associated with opioids, alcohol, tobacco, and other substances. (Lucas, P. et al. “Medical cannabis patterns of use and substitution for opioids & other pharmaceutical drugs, alcohol, tobacco, and illicit substances; results from a cross-sectional survey of authorized patients,” Harm Reduct J. 2019, 16(1), 9, doi: 10.1186/s12954-019-0278-6.). Intermittent use of cannabis was associated with a lower percentage of positive opioid relapses and improved medication compliance on naltrexone therapy (Church et al., “Concurrent substance use and outcome in combined behavioral and naltrexone therapy for opiate dependence,” Am J Drug Alcohol Abuse, 2001, 21, 441-452). Similarly, associations of intermittent or occasional cannabis use with improved retention in treatment for opioid dependence have also been reported (Ellner et al., “Marijuana use by heroin abusers as a factor in program retention,” J Consult Clin Psychol, 1977, 45:709-710, doi: 10.1037/0022-006X.45.4.709). Among opioid-dependent individuals undergoing naltrexone therapy, intermittent cannabis users (with 1-80% of UDS positive for cannabis) fared better than cannabis abstinent or consistent cannabis users in terms of treatment retention and medication compliance (Raby et al., “Intermittent marijuana use is associated with improved retention in naltrexone treatment for opiate-dependence,” Am J Addict, 2009, 18, 301-308, doi: 10.1080/10550490902927785).

CB1 receptors influence the rewarding effects of opiates. CB1 receptor anatagonists block the development of morphine-induced conditioned place preference in rats and mice (Chaperon et al., Involvement of central cannabinoid (CB1) receptors in the establishment of place conditioning in rats (1998). Psychopharmacology (Berl) 135(4), 324-332.), and mice lacking CB1 receptors display reduced morphine-induced CPP (Rice et al., “Conditioned place preference to morphine in cannabinoid CB1 receptor knockout mice”, Brain Res, 2002, 945(1), 135-138, doi: 10.1016/s0006-8993(02)02890-1) CB1 receptor knockout mice do not acquire heroin self-administration. SR141716A dose-dependently reduces heroin self-administration in rats (Navarro et al., “Functional interaction between opioid and cannabinoid receptors in drug self-administration”, J Neurosci, 2001, 21(14), 5344-5350, doi: 10.1523/JNEUROSCI.21-14-05344.2001).

Thus CB1 antagonists can be used to selectively treat conditioned place preference and prevent the genesis of opioid dependency (Manzanedo et al., “Cannabinoid agonist-induced sensitisation to morphine place preference in mice,” NeuroReport, 2004, 15, 1373-1377, doi: 10.1097/01.wnr.0000126217.87116.8c).

While medical cannabis is used widely in conjunction with opioids, as well as in conjunction with the administration of opiate-based narcotics for the treatment of chronic and acute pain, there is a need for a more nuanced dosage administration that will precisely administer the cannabinoids that will alleviate dependency, rather than cultivate it.

Stimulant Addiction

Recent evidence also supports the involvement of the endocannabinoid system in the neurobiological processes related to stimulant addiction. Addiction to psychostimulants such as cocaine, amphetamine, and its derivatives (i.e., methamphetamine, N-methyl-3,4-methylenedioxymethamphetamine (MDMA)) is a significant public health problem affecting many aspects of social and economic life, with between 16 and 51 million substance users worldwide (Oliere et al., “Modulation of the endocannabinoid system: vulnerability factor and new treatment target for stimulant addiction”, Front Psychiatry, 2013, 23, 4-109, doi: 10.3389/fpsyt.2013.00109).

In recent decades, development of new treatments for psychostimulant addiction has been a major focus of multidisciplinary research efforts, and has included molecular approaches, preclinical behavioral studies, and clinical trials.

Soria and colleagues observed that CB1 receptor deletion impairs the acquisition of cocaine self-administration by mice, and both genetic and pharmacological CB1 receptor blockade reduces the motivation for cocaine under a progressive ratio schedule of reinforcement (Soria et al., “Lack of CB I cannabinoid receptor impairs cocaine self-administration”, Neuropsychopharmacology, 2005, 30, 1670-1680, doi: 10.1038/sj.npp.1300707).

The CB1 receptor antagonist AM251 significantly attenuates the motivation for cocaine self-administration under a progressive ratio schedule of reinforcement (Xi et al., “Cannabinoid CB1 receptor antagonists attenuate cocaine's rewarding effects: experiments with self-administration and brain-stimulation reward in rats,” Neuropsychopharmacology, 2008, 33(7), 1735-1745, doi: 10.1038/sj.npp.1301552), reduces methamphetamine self-administration (Vinklerova et al., “Inhibition of methamphetamine self-administration in rats by cannabinoid receptor antagonist AM 251,” J Psychopharmacol, 2002, 16, 139-143, doi: 10.1177/026988110201600204) and attenuates cocaine-induced enhancement in the sensitivity to brain stimulation reward (Xi et al., “Cannabinoid CB1 receptor antagonists attenuate cocaine's rewarding effects: experiments with self-administration and brain-stimulation reward in rats,” Neuropsychopharmacology, 2008, 33(7), 1735-1745, doi: 10.1177/026988110201600204).

Orio and colleagues found that the CB I receptor influence on cocaine reward is enhanced by long periods of cocaine self-administration that result in progressive increases in cocaine intake (Orio et al., “A role for the endocannabinoid system in the increased motivation for cocaine in extended-access conditions”,J Neurosci, 2009, 29(15), 4846-4857, doi: 10.1523/JNEUROSCI.0563-09.2009). These observations show that neuroadaptations induced by extended cocaine exposure may recruit a CB I receptor involvement in a progressive escalation of drug intake that results from extended periods of cocaine use. [0076] Thus the targeted administration of selective CB1 receptor antagonists may be useful in the alleviation of chemical dependency associated with, and withdrawal from, psychostimulants.

Alcohol Addiction

Alcohol (ethanol) is a habit-forming drug that has been extensively studied for its relationships with the endocannabinoid signaling system (Hungund et al., “Are anandamide and cannabinoid receptors involved in ethanol tolerance? A review of the evidence,” Alcohol, 2000, 35: 126-133, doi: 10.1093/alcalc/35.2.126) This can be concluded from genetic studies that have proved a greater frequency for the appearance of a genetic polymorphism for the cannabinoid CB ] receptor in several subpopulations of alcoholic patients, in particular in alcoholics with severe withdrawal signs, such as delirium or seizures (Schmidt et al., “Association of a CB1 cannabinoid receptor gene (CNR1) polymorphism with severe alcohol dependence,” Drug Alcohol Depend, 2002, 65, 221-224, doi: 10.1016/S0376-8716(01)00164-8), or with antecedents of childhood attention deficit/hyperactivity (Ponce et al., “Association between cannabinoid receptor gene (CNR1) and childhood attention deficit/hyperactivity disorder in Spanish male alcoholic patients,” Mol. Psychiatry, 2003, 8, 466-467, doi: 10.1038/sj.mp.4001278), and also from biochemical studies that examined the effects of alcohol exposure on endocannabinoid signaling in laboratory animals or cultured nerve cells (Basavarajappa et al., “Chronic ethanol administration down-regulates cannabinoid receptors in mouse brain synaptic plasma membrane,” Brain Res, 1998, 79, 212-218, doi: 10.1016/s0006-8993(98)00175-9).

Chronic alcohol exposure modifies endocannabinoid levels in different brain regions, while pharmacological targeting of the endocannabinoid system has been reported to influence ethanol intake in laboratory animals.[¾] Pharmacological targeting of this system serves to reduce the incentive properties of alcohol, the signs of alcohol withdrawal, and/or the vulnerability to relapse. Mice treated with CB I antagonists showed a significant reduction in self-administered alcohol consumption (Colombo et al., “Suppressing effect of the cannabinoid CB1 receptor antagonist, SR 141716, on alcohol's motivational properties in alcohol-preferring rats,” Eur J Pharmacol, 2004, 498, 19-123, doi: 10.1016/j.ejphar.2004.07.069).

The instant invention provides for the administration of unique dosage forms of cannabinoids, including CB1 receptor antagonists like SR 141716 and other CBD antagonists, in the treatment of alcohol dependence.

Nicotine Addiction

CB1 knockout mice indicate a critical role of CB1 receptors in the rewarding effects of nicotine (Valjent et al., “Behavior al and biochemical evidence for interactions between Delta 9-tetrahydrocannabinol and nicotine,” Br J Pharmacol, 2002, 135(2), 564-578, doi: 10.1038/sj.bjp.0704479). Similarly, the administration of CB1 receptor antagonists like SR141716A have been successful in blocking the acquisition of nicotine-induced conditioned place preference in rats (Le Foil et al., “Rimonabant, a CB1 antagonist, blocks nicotine-conditioned place preferences,” Neuroreport, 2004, 15(13), 2139-2143, doi: 10.1097/00001756-200409150-00028; Forget et al.,“Cannabinoid CB1 receptors are involved in motivational effects of nicotine in rats,” Psychopharmacology (Berl), 2005, 181(4), 722-734); Cohen et al., “SR141716, a central cannabinoid (CB1) receptor antagonist, blocks the motivational and dopamine-releasing effects of nicotine in rats,” Behav Pharmacol, 2002, 13, 451-463).

Along with the more selective CB1 antagonist AM251 , SRI 41 76A dose-dependently reduces nicotine self-administration by rats (Cohen et al., “SR141716, a central cannabinoid (CB(1)) receptor antagonist, blocks the motivational and dopamine-releasing effects of nicotine in rats,” Behav Pharmacol, 2002, 13, 451-463).

The instant invention provides a measured dosage form capable of predictably administering precise, therapeutically effective amounts of cannabinoids, including, but not limited to, CB I receptor antagonists as a means of reducing nicotine dependency.

### Treatment of Adverse Effects Associated with Dependency and Withdrawal

High CBD intake relative to THC has been associated with lower scores on the positive dimensions of the CAPE (Community Assessment of Psychic Experiences) Scale, inhibition of psychotic symptoms, and reduced deficiencies in episodic memory (Englund et al., “Cannabidiol inhibits THC-elicited paranoid symptoms and hippocampal-dependent memory impairment,” J Psychopharmacol,” January 27(1), 2013, 19-27, doi: 10.1177/0269881112460109.). The present formulations present the advantage of an intentional dosage of synthetic or natural preparations with ideal pharmacology, to the exclusion of other cannabinoids, if desired, and their respective pharmacokinetic and pharmacodynamics influences. The “protective effects” afforded by extended or sustained release formulations can be harnessed by virtue of the present invention, along with the desired therapeutic effects of cannabidiols, either alone or in concert with therapeutically effective amounts of other cannabinoids.

The endocannabinoid system is intricately involved in the pathophysiology of depression, with CB1 receptors widely distributed in brain areas related to affective disorders, where expression is otherwise regulated by anti-depressants (Devane et al., “Determination and characterization of a cannabinoid receptor in rat brain,” Mol Pharmacol (1988) 34: 605-613; Hill and Gorzalka, Is there a role for the endocannabinoid system in the etiology and treatment of melancholic depression?” Behav Pharmacol, 2005, 16, 333-352; Hill et al., “Regional alterations in the endocannabinoid system in an animal model of depression: effects of concurrent antidepressant treatment,” J Neurochem, 2008, 106, 2322-36, doi: 10.1111/j.1471-4159.2008.05567.x).

Administration of CBD achieves characteristic effects of induced anti-psychotic and anxiolytic activity in subjects, and also attenuates the development of stress-induced behavior al consequences (Guimaraes et al., “Antianxiety effect of cannabidiol in the elevated plus-maze,” Psychopharmacology,1990, 100, 558-559; Resstel et al., “Effects of cannabidiol and diazepam on behavior al and cardiovascular responses induced by contextual conditioned fear in rats,” Behav Brain Res, 2006, 172(2), 294-98, doi: 10.1016/j.bbr.2006.05.016; Resstel et al., “5-HT1A receptors are involved in the cannabidiol-induced attenuation of behavior al and cardiovascular-induced attenuation of behavior al and cardiovascular responses to acute restraint stress in rats,” Br J Pharmacol, 2009, 156(1), 181-88, doi: 10.1111/j.1476-5381.2008.00046.x; Zuardi et al., “Action of cannabidiol on the anxiety and other effects produced by delta 9-THC in normal subjects,” Psychopharmacology, 1982, 76, 245-50).

The instant invention is important and useful because CBD has a favorable profile in a model predictive of antidepressant-like activity in comparison to antidepressants, but such effects are only attainable at precise dosages, with smaller or higher doses producing no effect (Porsolt et al., “Depression: a new animal model sensitive to antidepressant treatment,” Nature, 1977, 266, 730-32, doi: 10.1038/266730a0; Zanelati et al., “Antidepressant-like effects of cannabidiol in mice: possible involvement of 5-HT1A receptors,” Br J Pharmacol, 2010, 159, 122-28, doi: 10.1111/j.1476-5381.2009.00521.x). The present invention provides formulations which are capable of administering dosages in accordance with the most clinically appropriate pharmacokinetic and pharmacodynamic profile in order to achieve desired therapeutic effects in a patient presenting with a specific pathophysiology, such as major depressive disorder.

Sleep disturbances are a common adverse effect associated with withdrawal from chemical dependency, and for which certain cannabinoids can provide relief. The chemistry of CBD has been examined, and its central nervous system (CNS) pharmacological properties, including its anticonvulsant, anxiolytic, and sedative effects, have been documented (Chesher et al., (1975) “Interaction of D⁹-tetrahydrocannabinol and cannabidiol with phenobarbitone in protecting mice from electrically induced convulsions,” J. Pharm. Pharmacol. 27:608-609; Pickens, “Sedative activity of Cannabis in relation to its delta′-trans-tetrahydrocannabinol and cannabidiol content” Br. J. Pharmacol, 1981, 72, 649-656; Russo et al., “A tale of two cannabinoids: the therapeutic rationale for combining tetrahydrocannabinol and cannabidiol,” Med. Hypotheses, 2006, 66, 234-246, doi: 10.1016/j.mehy.2005.08.026).

It has been shown that CBD improves sleep in individuals suffering from insomnia (Carlini et al., “Hypnotic and antiepileptic effects of cannabidiol,” J. Clin. Pharmacol, 1981, 21 (suppl 8-9), 417S-427S, doi: 10.1002/j.1552-4604.1981.tb02622.x). It has been successfully employed to block anxiety-induced REM sleep alteration via its anxiolytic effects (Hsiao et al., “Effect of cannabidiol on sleep disruption induced by the repeated combination tests consisting of open field and elevated plus-maze in rats,” Neuropharmacol (2012) 62: 373-84). Other studies have exhibited clinically significant improvements in sleep in subjects suffering from post-traumatic stress-related insomnia, even when subjects received no pharmaceutical medications to treat sleep disorders aside from cannabidiol oil (Shannon, “Effectiveness of cannabidiol oil for pediatric anxiety and insomnia as part of posttraumatic stress disorder: a case report,” Perm J 2016, 20(4), 16-005, doi: 10.7812/TPP/16-005). The systemic acute administration of CBD increases total sleep time in subjects, in addition to increasing sleep latency in the light period of the day of administration (Chagas et al., “Effects of acute systemic administration of cannabidiol on sleep-wake cycle in rats,” J. Psychopharmacol, 2013, 27, 312-16, doi: 10.1177/0269881112474524).

Treatment of Drug-Seeking (Relapse)

Both positive and negative memories and conditioned cues associated with drug use perpetuate drug-seeking behavior and the continued cycle of abuse. Drug exposure produces powerful interoceptive effects that become associated with environmental cues, such that these cues alone can induce craving and promote relapse following periods of abstinence (Carter et al., “Cue-reactivity and the future of addiction research,” Addiction, 1999, 94, 349-51, doi: 10.1046/j.1360-0443.1999.9433273.x). In addition to conditioned drug memories, acute exposure to a preferred drug or pharmacologically related agent (that is, drug priming) and stressful events can precipitate relapse (Koob et al., “Stress, dysregulation of drug reward pathways, and the transition to drug dependence,” Am J Psychiatry, 2007, 164, 1149-1159, doi: 10.1176/appi.ajp.2007.05030503).

Animal models of relapse demonstrate an important cannabinoid influence on the reinstatement of extinguished drug-seeking and drug-taking behaviors. Taco J. DeVries and Anton N. M.Schoffelmeer, “Cannabinoid CB1 receptors control conditioned drug seeking,” Trends Pharmacol. Sci. 2005, 26, 420-426 https://doi.org/10.1016/j.tips.2005.06.002. CB1R blockade attenuates relapse-like behavior in rats, thus paving the way for numerous studies demonstrating a potent influence of CB1 R signalling on relapse-like behavior induced both by drug exposure and by drug-paired conditioned cues across multiple classes of abused drugs (Fattore et al., “An endocannabinoid mechanism in relapse to drug seeking: a review of animal studies and clinical perspectives,” Brain Res, 2007, Rev. 53, 1-16, doi: 10.1016/j.brainresrev.2006.05.003.).

CB1 receptor antagonism attenuates drug-primed, cue-induced and some forms of stress-induced reinstatement of cocaine- and methamphetamine-seeking behavior in rats (Serrano et al., “Endocannabinoid influence in drug reinforcement, dependence and addiction-related behaviors,” Pharmacol, Ther. 2011, 132, 215-241, doi: 10.1016/j.pharmthera.2011.06.005). Thus, CB1 R signaling modulates drug-seeking for various pharmacologically distinct drugs. There is also evidence that CB1 receptor antagonism blocks both cue- and priming-induced reinstatement of seeking behavior for non-drug rewards, such as sucrose and corn oil (De Vries et al., “Suppression of conditioned nicotine and sucrose seeking by the cannabinoid-1 receptor antagonist SR141716A,” Behay. Brain, 2005, Res. 161, 164-168, doi: 10.1016/j.bbr.2005.02.021

Formulations

CBD and THC in the pure state are solids. Pure CBD has a reported melting point of 62-66° C. (Chemspider). Pure THC has a predicting melting point of 160° C. (Chemspider). Both of these cannabinoids are poorly soluble in water and most formulations to date have been liquid or oil formulations, including tinctures and gelcaps. In view of the intense interest in these drugs, and the likely legalization and rescheduling of cannabis, new formulations of cannabinoids are urgently needed.

This invention involves dry granulations of cannabinoids. Granulation is the process of particle enlargement by agglomeration. Granulation is one of the most significant unit operations in the production of pharmaceutical dosage forms, mostly tablets and capsules. Granulation transforms fine powders into free-flowing, dust-free granules that are easy to compress. (Srinivasan Shanmugam, Granulation techniques and technologies: recent progresses, Bioimpacts, 2015, 5(1), 55-63, doi: 10.15171/bi.2015.04 PMCID: PMC4401168).

In particular, this invention involves dry granulation methods, i.e., granulates formed without the use of a solvent in the granulation process. Compressible dosage forms made by dry granulation are known in the art, from e.g., WO 2009/135948A2 and U.S. Pat. No. 4,439,453. Cannabinoid formulations in oral dosage forms are disclosed in e.g., U.S. 2018/0221332A1, U.S. 2016/0143972 A1, and U.S. 20180271827A1. However, none of these references disclose dry granulations of cannabinoids and high drug loadings as provided by the instant invention.

### SUMMARY OF THE INVENTION

In an embodiment, this invention discloses a compressible pharmaceutical composition comprising a cannabinoid and at least one excipient, wherein the cannabinoid comprises 5%-90% w/w of the composition, and wherein the excipient is a compressible pharmaceutical binder, and wherein the composition is a dry powder of 20 mesh or smaller particle size. The cannabinoid may be selected from CBD, THC, or another solid cannabinoid. In an embodiment, the compressible pharmaceutical binder is selected from microcrystalline cellulose (MCC), silicified microcrystalline cellulose (SiMCC), hydroxypropyl cellulose (HPC), a glycol, a polaxamer, lactose, mannitol, polyvinyl pyrrolidone (PVP), a starch, or a matrix-forming polymer such as a polyvinylpyrrolidone-vinyl acetate copolymer; a polyvinylcaprolactam, polyvinyl acetate, and polyethylene glycol 6000 copolymer; or an ethylene oxide and propylene oxide copolymer. In an embodiment, the composition may include a compressible pharmaceutical disintegrant is selected from a starch, croscarmellose sodium, cross-linked polyvinyl pyrrolidone (crospovidone), or a cellulose. In an embodiment, the compressible pharmaceutical lubricant is selected from magnesium stearate, stearic acid or a natural gum.

In embodiment, this invention provides a process for the preparation of a compressible pharmaceutical composition, comprising the steps of mixing a dry powdered cannabinoid with magnesium stearate and a binding excipient in a twin shell blender to form a uniform mixed blend; slugging the uniformly mixed blend in a tablet press to form slugs, breaking the slugs with an oscillating mill and passing the granulation through a 20-mesh screen to obtain a 20-mesh granulate.

In an embodiment, this invention provides a process for the preparation of a compressible pharmaceutical composition, comprising the steps of mixing a dry powdered cannabinoid with magnesium stearate and a binding excipient in a twin shell blender to form a uniform mixed blend, and compacting the uniformly mixed blend in a rotary compaction press to form a ribbon, and pulverizing the ribbon with an oscillating mill and passing the pulverized material through a 20-mesh screen to obtain a 20-mesh granulate.

In an embodiment, this invention provides a process for the preparation of a compressible pharmaceutical composition, comprising the steps of feeding a dry powdered cannabinoid and a matrix-forming polymer and optionally additional excipients into a hot-melt extrusion machine, wherein the hot-melt extrusion machine has at least one Archimedes screw in a zone heated to a sufficient temperature to melt the matrix forming polymer, wherein the screw in the hot zone mixes the cannabinoid, matrix-forming polymer, and optional additional excipients into a uniformly blended plastic mixture, and forcing the plastic mixture through an orifice to form an extrudate. The extrudate is cooled and formed into a granulate by milling the extrudate to a desired particle size.

### DETAILED DESCRIPTION

Disclosed herein is a compressible pharmaceutical composition comprising a cannabinoid and a compressible excipient formed by a dry granulation method, and suitable for compression into tablets. By the term “dry granulation” it is meant that no solvent is used in the granulation process, such as water, ethanol, or another solvent or solvent blend. Thus, in an embodiment, the composition is a pharmaceutical intermediate suitable for further processing into oral dosage forms, such as immediate release, controlled release formulations or orally dissolving formulations. Cannabinoids include CBD, THC, or synthetic variants that have medical or recreational pharmaceutical value. Compressible excipients include microcrystalline cellulose (MCC), silicified microcrystalline cellulose (SiMCC), hydroxypropyl cellulose (HPC), lactose, mannitol, or a starch. In an embodiment, the composition is 70%-90% by weight of the cannabinoid. In an embodiment, additional ingredients may be included, such as magnesium stearate. In an embodiment, this material has a particle size of 20 mesh or less.

Particular advantages of the instant invention compared to conventional formulations are the dry granulation methods, and the high drug loadings that can be achieved relative to prior art methods.

In some embodiments, a matrix-forming agent for hot-melt extrusion may be used. These matrix forming agents include various co-polymers, for example Kollidon® VA 64, a polyvinylpyrrolidone-vinyl acetate copolymer; Soluplus®, a co-polymer of polyvinylcaprolactam, polyvinyl acetate, and polyethylene glycol 6000, available from BASF; and Kolliphor® P 188, a poloxamer (BASF). Other Kolliphor grades are available also and are within the scope of this invention. Poloxamers are copolymers of ethylene oxide and propylene oxide. All of these matrix forming agents are compressible and are supplied as free flowing powders.

In an embodiment, this material is intended to be mixed with other materials that can be formed into tablets by a compression method. For example, the inventive intermediate can be blended with diluents, controlled release agents, fillers, disintegrants, and the like known in the art of tablet formulations.

In an embodiment, a very high concentration of cannabinoid is desirable, such as 70% to 90% by weight. This type of intermediate (composition) may give maximal flexibility to formulators to blend the inventive intermediate with other materials to achieve a desired effect, for example a controlled-release formulation or a quick-release formulation, such as a sublingual orally dissolving tablet. In an alternative embodiment, the cannabinoid concentration may be lower. A concentration as low as 5% is envisioned by this invention. Thus, an embodiment may have 5% to 30% by weight of cannabinoid, or 30% to 50% by weight of cannabinoid, or 50% to 90% by weight of cannabinoid, wherein any of the embodiments mentioned in this paragraph are made by the dry granulation of this invention.

In the utility of the inventive composition as a pharmaceutical intermediate, low concentration compositions may also be desirable, allowing tablet manufacturers to minimize additional processing by directly using a low concentration material having e.g., 5-10% w/w of a cannabinoid. For example, it may be possible to compress the inventive composition into a tablet with no additional ingredients, or only 1-2% of additional ingredients, such as a coloring agent, fragrance, artificial sweetener or other flavoring agent. CBD doses are typically in the 1 mg to 10 mg per dose range, so a 100 mg tablet (total tablet weight) made from the inventive mixture having 10% w/w CBD would provide a tablet with a 10 mg dose.

The cannabinoid may be any pharmaceutically active agent extracted from cannabis plant material, or a chemically related synthetic variant thereof. In an embodiment, the cannabinoid may be cannabidiol, also termed herein CBD, or (−)-trans-Δ⁹-tetrahydrocannabinol, referred to herein as THC.

### Manufacturing Processes

In an embodiment, this invention provides a process for manufacturing the compressible pharmaceutical composition.

In an embodiment, the inventive intermediate is formed by a dry granulation method. Several dry granulation methods are known in the art, for example slugging, roller compaction, and hot-melt extrusion. In the slugging and roller compaction methods, two or more ingredients are subjected to a compression force (also termed “compaction”), so the ingredients form an intimate uniformly distributed mixture of the ingredients without the use of a solvent. In the hot-melt extrusion method, ingredients with a meltable polymer are mixed in a hot zone in a machine with an Archimedes screw and extruded and milled to a desired particle size. Thus, hot-melt extrusion does not involve the use of a solvent and can be termed a dry-granulation method. Dry granulation methods do not use a solvent such as water, alcohol, or some other solvent to assist in the uniform blending of ingredients. For this reason, dry granulation may have the advantage of lower costs of not requiring a solvent that must be safely disposed of.

Slugging Dry Granulation

The slugging dry granulation method employs a tablet press to compact a mixture into crude tablets called “slugs” that are then broken apart to a desired size by milling.

In a slugging process, a mixture of a cannabinoid such as pure CBD in a solid powdered form is blended in a V-blender with a compressible excipient and any other ingredients, such as a disintegrant or lubricant such as magnesium stearate. The blend is then compressed into tablets on a tablet press. These tablets are not intended for consumption. The tablet press may use any of several available punches, such as a flat face punch, and be anywhere from 7-20 mm along the longest dimension. The compression force is not highly critical at this stage, and may be from about 5-30 KN. The resulting tablets are termed “slugs.” The slugs are then milled and sieved. In an embodiment, there may be sequential milling and sieving steps.

Roller Compaction Dry Granulation

In an embodiment of this invention, roller compaction may be used to form the granulate of this invention. Roller compaction processes employ a roller compaction machine. Leading manufacturers are Gerteis, Komareck, and others. These devices typically have settings for the gap and roller speed, which determine the compaction force. The method is usually used to produce a ribbon or briquets of the compressed material. Either product may be milled to a desired particle size.

Roller compaction is a dry-granulation method employing two counter-rotating rollers that compact a mixture forcing a feed between the rollers. Typically, roller compaction employs the following steps: powdered material is conveyed to the compaction area, e.g. with a screw feeder. The powder is compacted between two counter-rotating rollers with applied forces and milling the resulting compact to a desired particle size distribution. Preferably, during roller compaction the powdered material is transported by gravity forces or screws into a gap between two counter rotating rolls. Within the gap the material is densified to a compact by the force transmitted from the rolls. Depending on the surface of the used rolls different types of compacts may be generated (e.g. ribbons, briquettes). Using knurled or smooth surfaces of the rolls a compact band is produced, which is called ribbon. In a second step, the grinding step, the produced compacts may be grinded through a sieve to produce granules.

Hot-melt Extrusion

In an embodiment, hot-melt extrusion may be used to manufacture the inventive compositions (Rina Chokshi et al., “Hot-Melt Extrusion Technique: A Review,” Iranian J. Pharm, 2004, 3, 3-16). In a hot-melt process (also referred to in the literature as a “melt process”), the active agent is mixed with a matrix-forming polymer that melts at elevated temperature, which typically ranges from about 60° C. to 160° C. The active agent and polymer are fed into a hot-melt extruder which comprises one or two Archimedes screws in a hot zone that mixes the materials and melts the matrix-forming polymer to form a plastic mixture. A barrel section in the extruder houses the screw and is heated to the desired temperature as the screw conveys and mixes the material. A variety of screws with different pitches may be selected to achieve the desired mixing and conveyance. Some extruders have twin screws in the mixing process. At the end of the barrel, the melt is forced by the screw(s) through an orifice that may make a ribbon, rod, or other extruded shape. The extrudate is then cooled and milled to a desired mesh size to provide a granulate according to this invention.

A particular advantage to hot-melt extrusion is that the percentage of active agent (active pharmaceutical ingredient, or API) may be fairly high, with 40-60% of active agent can be used, with about 60-40%% of matrix forming polymer. This gives the kind of concentrated product that is desirable in many embodiments of this invention, that can be used in blends for tableting and other formulations as discussed in this disclosure. Moreover, CBD and THC can easily withstand the elevated temperatures in hot-melt extrusion without degradation. Appropriate temperatures and matrix-forming polymers should be selected to prevent thermal degradation in the hot-melt apparatus.

Some common matrix-forming polymers that may be useful in this invention include Kollidon® VA 64, a polyvinylpyrrolidone-vinyl acetate copolymer (BASF); Soluplus®, a co-polymer of polyvinylcaprolactam, polyvinyl acetate, and polyethylene glycol 6000, available from BASF; and Kolliphor® P 188, a poloxamer (BASF). Other Kolliphor grades are available also and are within the scope of this invention. These materials all act as matrix forming agents and binders and are compressible.

Melt Granulation

Another process that may be used is melt granulation. See Desai, et al., “Melt granulation: An alternative to traditional granulation techniques,” Indian Drugs. 2013. 50. 5-13; see also T. Listro, https://www.pharmasalmanac.com/articles/twin-screw-melt-granulation-as-a-platform-technology-for-continuous-manufacturing. Melt granulation is a size enlargement process in which the addition of a binder that melts or softens at relatively low temperatures (about 60° C.) is used to achieve agglomeration of solid particles in the formulation. These are lower temperatures than HME, but the equipment (single or twin screw extruders) may be the same. The process utilizes materials that are effective as granulating agents when they are in the softened or molten state. This process is particularly useful for formulations of lipophilic drug products. This technique can produce cannabinoid drug loadings as high as 80-90%.

In this method, a combination of a dry powdered cannabinoid and a matrix-forming polymer and optionally additional excipients into a hot-melt extrusion machine, wherein the hot zone of the extruder is kept at a temperature of 60-120° C., which is sufficient to soften but not melt the polymer. Under the action of the screw, a granulate is formed that is forced through the orifice of the extruder. The granulate can then be milled to a desired particle size, for example 20 mesh.

The approximately 20 mesh particles of any of these methods may be used in a tablet formulation to produce cannabinoid tablets.

### EXAMPLE 1

This is an example of the slugging method so produce a suitable compressible granulate.

| Ingredient | % w/w | g/batch |
|---|---|---|
| CBD | 75.00 | 22.50 |
| SiMCC | 24.50 | 7.35 |
| Mg Stearate | 0.50 | 0.15 |
| Total | 100.00 | 30.00 |

1. CBD, Cellulose and Mg Stearate were blended in a twin shell mixer for 5 minutes.
2. The blend was slugged blend using 10 mm flat face punches with 20 kN pressure.
3. The slugs were milled with an oscillating mill equipped with a 12-mesh screen
4.The granulate was milled further with an oscillating mill and passed through a 20-mesh screen to give a 20-mesh granulate.

This experiment yielded a satisfactory granulate that can be compressed into tablets.

### EXAMPLE 2

This example employs a roller-compactor method to make the inventive dry granulate. The following ingredients are used.

| Ingredient | % w/w | g/batch |
|---|---|---|
| CBD | 75.00 | 22.50 |
| SiMCC | 24.50 | 7.35 |
| Mg Stearate | 0.50 | 0.15 |
| Total | 100.00 | 30.00 |

1. Blend CBD, Cellulose and Mg Stearate in Twin shell mixer for 5 minutes.
2. The blend is compressed on a Gerteis Mini-Pactor® pilot scale roller compactor to form a ribbon, using the following parameters:

| | |
|---|---|
| Press Force | 1-20 kN/cm |
| Roller Speed | 1-30 rpm |
| Gap | 1-6 mm |

3. The ribbon is broken up with an oscillating mill equipped with a 20-mesh screen, to give the granulate. This experiment yields a 20 mesh or less granulate that can be compressed into tablets.

### EXAMPLE 3

This example employs hot-melt extrusion to form a dry granulate.

| Ingredient | % w/w | g/batch |
|---|---|---|
| CBD | 39.50 | 11.85 |
| Soluplus ® | 60.00 | 18.00 |
| Mg Stearate | 0.50 | 0.15 |
| Total | 100.00 | 30.00 |

Soluplus is a co-polymer of polyvinylcaprolactam, polyvinyl acetate, and polyethylene glycol 6000, in a ratio of 57/30/13, available from BASF (https://pharmaceutical.basf.com/en/Drug-Formulation/Hot-melt-extrusion.html, visited Jul. 17, 2019). Soluplus exhibits both matrix-forming and solubilization properties. This material may be blended with active ingredient (e.g., CBD) and any other excipient such as magnesium stearate. The blended powder is fed through a hot-melt extrusion apparatus at 120° C., at a rate of 1 kg/h, with a 1 kneading block with 5×0.25 D kneading elements at 90°, screw speed 200 rpm, and torque 0.5 Nm. The method produces an extrudate that can be milled to a desired size such as 20 mesh with an oscillating sieve.

### EXAMPLE 4

This example employs hot-melt extrusion to form a dry granulate of THC.

| Ingredient | % w/w | g/batch |
|---|---|---|
| THC | 60.00 | 18.00 |
| Soluplus ® | 39.50 | 11.85 |
| Mg Stearate | 0.50 | 0.15 |
| Total | 100.00 | 30.00 |

Soluplus may be blended with THC ((−)-trans-Δ⁹-tetrahydrocannabinol) as the active ingredient and any other excipient such as magnesium stearate. The blended powder is fed through a hot-melt extrusion apparatus at 120° C., at a rate of 1 kg/h, with a 1 kneading block with 5×0.25 D kneading elements at 90°, screw speed 200 rpm, and torque 0.5 Nm. The method produces an extrudate that can be milled to a desired size such as 20 mesh with an oscillating sieve. This material can be formulated into pharmaceutical compositions with recreational or medical value.

### EXAMPLE 5

This example employs the melt granulation technique to form a dry granulate of a cannabinoid. This example can be used with THC or CBD, or a combination of the two, or include another cannabinoid.

| Ingredient | % w/w | g/batch |
|---|---|---|
| THC or CBD | 89.50 | 26.85 |
| Soluplus ® | 10.00 | 3.00 |
| Mg Stearate | 0.50 | 0.15 |
| Total | 100.00 | 30.00 |

Soluplus may be blended with THC or CBD, or a combination thereof, as the active ingredient and any other excipient such as magnesium stearate. The blended powder is fed through a hot-melt extrusion apparatus at 80° C., at a rate of 1 kg/h, screw speed 100 rpm, and torque 0.5 Nm. The method produces a granulate that can be milled to a desired size such as 20 mesh with an oscillating sieve. This material can be formulated into compressible pharmaceutical compositions with recreational or medical value.

1. A compressible pharmaceutical composition comprising a cannabinoid and at least one excipient wherein the composition is formed without the use of a solvent, wherein the cannabinoid loading comprises 25%-90% w/w of the composition, and wherein the excipient is a compressible pharmaceutical binder, and wherein the composition is a dry powder of 20 mesh or smaller particle size.

2. The composition of claim 1 wherein the cannabinoid is selected from CBD or THC or a mixture thereof.

3. The composition of claim 1, wherein the compressible pharmaceutical binder is selected from microcrystalline cellulose (MCC), silicified microcrystalline cellulose (SiMCC), hydroxypropyl cellulose (HPC), lactose, mannitol, or a starch.

4. The composition of claim 1, wherein the compressible pharmaceutical binder is selected from a polyvinylpyrrolidone-vinyl acetate copolymer, a polyvinylcaprolactam, polyvinyl acetate, and polyethylene glycol copolymer, and a ethylene oxide and propylene oxide copolymer.

5. (canceled)

6. The composition of claim 1, wherein the cannabinoid is 25%-50% w/w.

7. The composition of claim 1, wherein the cannabinoid is 40%-60% w/w

8. The composition of claim 1, wherein the cannabinoid is 50%-90% w/w.

9. The composition of claim 1, wherein the cannabinoid is 50% w/w.

10. The composition of claim 1, wherein the cannabinoid is 75% w/w.

11. The composition of claim 1, wherein the cannabinoid is CBD present in 75% w/w of the composition.

12. (canceled)

13. A process for the preparation of a compressible pharmaceutical composition, comprising the steps of:
a. Mixing a dry powdered cannabinoid with magnesium stearate and a binding excipient in a blender for 5-30 minutes to form a uniform mixed blend;
b. Slugging the uniformly mixed blend in a tablet press using 7-20 mm punches with 5-30 kN compression pressure to form slugs;
c. Breaking the slugs with an oscillating mill equipped with 12-mesh screen;
d. Passing the granulation through a 20-mesh screen using an oscillating mill to obtain a 20-mesh granulate; and
e. wherein the total weight percent of the cannabinoid is 25-90% w/w of the granulate.

14. The process of claim 13, wherein the cannabinoid is selected from CBD or THC or both.

15. The process of claim 13, wherein the binding excipient selected from microcrystalline cellulose (MCC), silicified microcrystalline cellulose (SiMCC), hydroxypropyl cellulose (HPC), lactose, mannitol, or a starch.

16. (canceled)

17. The process of claim 13, wherein the weight percent of the binder is 19.5% to 50%.

18. The process of claim 13, wherein the amount of magnesium stearate is 0.25% to 2.0% w/w of the composition.

19. The process of claim 13, wherein the amount of magnesium stearate is about 0.5% w/w of the composition.

20. A process for the preparation of a compressible pharmaceutical composition, the process comprising the steps of:
a. Mixing 75.00% by weight of dry powdered cannabidiol with 0.50% by weight of Magnesium Stearate and 24.500% by weight of a binding excipient in a twin shell blender for at least 5 minutes;
b. Slugging the uniformly mixed blend in a tablet press using 10 mm flat face punches with 20 kN compression pressure;
c. Breaking the slugs with an oscillating mill equipped with 12-mesh screen;
d. Passing the granulation through a 20-mesh screen using an oscillating mill to obtain a 20-mesh granulate.

21. (canceled)

22. A process for the preparation of a compressible pharmaceutical composition, comprising the steps of:
a. Mixing a dry powdered cannabinoid with magnesium stearate and a binding excipient in a blender for 5-30 minutes to form a uniform mixed blend;
b. compacting the uniform mixed blend in a roller compacter with 5-30 kN pressure to form a ribbon;
c. Breaking the ribbon with an oscillating mill equipped with 20-mesh screen to obtain a 20-mesh granulate; and
d. wherein the total weight percent of the cannabinoid is 25-90% w/w of the granulate.

23-27. (canceled)
